(19) United States
(12) Patent Application Publication
(10) Pub. No.: US 2013/0261481 A1
(43) Pub. Date: Oct. 3, 2013

(54) SYSTEMS AND METHODS FOR ST SEGMENT STABILITY DISCRIMINATION DURING CARDIAC ISCHEMIA DETECTION FOR USE WITH IMPLANTABLE MEDICAL DEVICES

(71) Applicants: Kathleen Kresge, Minneapolis, MN (US); Carol Hudgins, Crestwood, KY (US); Peter Chelius, Franklin, WI (US)

(72) Inventors: Kathleen Kresge, Minneapolis, MN (US); Carol Hudgins, Crestwood, KY (US); Peter Chelius, Franklin, WI (US)

(73) Assignee: PACESETTER, INC., Sylmar, CA (US)

(21) Appl. No.: 13/434,546

(22) Filed: Mar. 29, 2012

(51) Int. Cl. A61B 5/0456

(52) U.S. Cl. 600/521

(57) ABSTRACT

Techniques are provided for discriminating episodes of cardiac ischemia indicated based on shifts in ST segment elevation from false detections due to atrial fibrillation (AF) or other confounding factors such as premature ventricular contractions (PVCs.) In an example for use with a single-chamber device, in response to a possible ischemic event, the single-chamber device assesses ventricular stability based an examination of ventricular intracardiac electrogram (IEGM) signals. If the ventricular IEGM is unstable due to paroxysmal AF or frequent PVCs, the ischemic event is rejected as a false detection. Otherwise, the device responds to the event by, for example, generating warning signals, recording diagnostic data or controlling device therapy. The stability discrimination techniques are particularly advantageous for use within single-chamber devices that lack automatic mode switching but are also beneficial within at least some dual-chamber devices or multi-chamber systems.

### FIELD OF THE INVENTION

The invention relates to implantable medical devices such as pacemakers and implantable cardioverter/defibrillators (ICDs) and, in particular, to techniques primarily for use with single-chamber devices for detecting cardiac ischemia events such as acute coronary syndrome (ACS) events.

### BACKGROUND OF THE INVENTION

Cardiac ischemia is a condition whereby heart tissue does not receive adequate amounts of oxygen and is usually caused by a blockage of an artery leading to heart tissue. If sufficiently severe, cardiac ischemia results in an acute myocardial infarction (AMI) or other ACS event. With AMI, a substantial portion of heart muscle ceases to function because it no longer receives oxygen, usually due to significant blockage of the coronary artery, a condition that may be fatal to the patient. However, many episodes of cardiac ischemia are not sufficiently serious to cause actual permanent injury to the heart tissue. Nevertheless, it is desirable to detect such instances of “silent” cardiac ischemia.

Various techniques have been developed for analyzing morphological features of intracardiac electrogram (IEGM) signals sensed by implantable medical devices in an effort to detect ischemia. In particular, some IEGM-based cardiac ischemia detection techniques seek to detect ischemia by identifying changes in the elevation of the ST interval of the IEGM that can occur during cardiac ischemia. The ST interval represents the portion of the cardiac signal between ventricular depolarization (also referred to as an R-wave or QRS complex) and ventricular repolarization (also referred to as a T-wave). The elevation of the ST interval can shift due to cardiac ischemia or other factors.

Accordingly, many state-of-the-art devices are equipped with an ST monitoring feature that monitors ST segment elevation to detect shifts indicative of ischemia. Issues, however, can arise when ST monitoring is employed in single-chamber pacemakers and ICDs (e.g. devices with a right ventricular (RV) lead but no right atrial (RA) lead), particularly in the presence of paroxysmal atrial fibrillation (AF) or frequent premature ventricular conducted beats (PVCs). Both AF and PVCs can cause false positive detections of cardiac ischemia or related conditions because accurate detection of shifts in ST segment elevation becomes challenging in the presence of AF and PVCs. In this regard, note that AF is the most common arrhythmia. According to the Framingham Heart Study (Kannel et al., “Epidemiological Features of Chronic Atrial Fibrillation,” NEJM. 1982;306:018-22), AF has a prevalence of four percent in the adult population. As the patient population continues to age, the prevalence of this arrhythmia rises as well, from less than 0.05 percent in patients 25 to 35 years of age to more than five percent in patients over 69 years of age. (Furberg et al. “Prevalence of AF in Elderly Subjects,” Am J Cardiol. 1994;74:236-241.)

A significant proportion of false positive detections of ischemic events are the result of ST segment changes arising with paroxysmal AF and PVC. In dual-chamber devices that have an RA lead for detecting AF, ST-based ischemia monitoring is suspended during an Automatic Mode Switch (AMS) triggered when the atrial rate exceeds a threshold indicative of AF. This prevents false positive ischemia detections that might otherwise occur due to ST shifts caused by abnormally conducted beats. However, single-chamber devices lack AMS and so false positive detections could occur within such devices resulting in improper device operation (or ST monitoring would need to be disabled within patients known to exhibit paroxysmal AF.) Although false detections can be avoided by disabling ST monitoring in patients who manifest paroxysmal AF, it would be preferable to provide an ST monitoring feature for use in single-chamber devices that would allow for ST monitoring within such patients while nevertheless avoiding false positives. At least some aspects of the invention are directed to this end.

### SUMMARY OF THE INVENTION

In an exemplary embodiment, a method is provided for use with an implantable medical device for implant within a patient for discriminating the stability of ST segments during ischemia detection. Briefly, a ventricular intracardiac electrogram (IEGM) signal is sensed and ST segments are identified therein. The ST segments are analyzed to detect an indication of a possible cardiac ischemic event within the patient. In response to the event, the stability of the ventricular IEGM is assessed. If the ventricular IEGM is unstable due, e.g., to paroxysmal AF or frequent PVCs, the event is rejected as a false detection. Otherwise, the device responds to the ischemic event by, for example, generating warning signals to alert the patient or caregiver, recording diagnostic data indicative of the event or controlling device therapy. The method is particularly advantageous for use within single-chamber pacemakers and ICDs that lack AMS but may also be beneficial within dual-chamber or multi-chamber devices. For example, if a problem with an RA lead prevents it from reliably tracking the atrial rate, AMS may be deactivated within the device while ST monitoring continues to detect ischemia while exploiting the aforementioned stability discrimination procedure. As another example, if the patient develops chronic AF, AMS may likewise need to be deactivated.

In an illustrative embodiment where the implantable device is a single-chamber pacer/ICD, the ST monitor of the device analyzes ST segments to detect an indication of a possible ischemic event by detecting shifts in ST segment elevation over time. For example, the ST monitor may track the most recent ninety seconds of ST segment data to detect a “shifted set” of segments. If a shifted set is detected, the device switches to more frequent monitoring (such as every thirty seconds) in an attempt to confirm the shift. Three consecutive shifted sets are needed before the ST monitor indicates detection of an ST shift event (i.e. a possible ischemia episode.) For each of the shifted sets, the device assesses the stability of the ventricular IEGM. For example, the device may assess ventricular stability by comparing shifted set stability (e.g. 2nd longest R-R interval minus 2nd shortest R-R interval) to an average from a baseline extraction stability (e.g. average of 2nd longest minus 2nd shortest from six-hour baseline extractions taken from the previous seventy-two hours.) In another example, the device compares R-R interval stability for the latest shifted set to an averaged baseline R-R interval stability obtained by calculating an average ventricular R-R interval stability value every six hours within baseline extractions from the previous seventy-two hours of the patient's normally conducted intrinsic sinus beats for each of several heart rate zones.

The device then compares the measured ventricular stability against a stability threshold set based on a current heart rate zone. The device rejects the ischemic event as a false detection (due to paroxysmal AF or other irregularities) if all three of the shifted sets (or, depending upon device programming, just two of the three sets) have a measured ventricular stability value crossing a threshold indicative of poor stability. For example, if the stability calculation indicates that the shifted sets are unstable and the value is greater than a programmed threshold (specified in milliseconds (ms) or as a percentage value), the ST episode is considered irregular/AF and no ST Episode is declared. That is, the event is rejected. If the event is rejected as a false detection, the device resets the ST monitor to ninety-second monitoring. If not rejected, the device responds to the ST event by taking appropriate and pre-programmed action, such as generating warning signals, modifying pacing therapy, etc.

In another illustrative embodiment, the device additionally tracks PVCs during the assessment of ventricular stability. PVCs may be detected, for example, based on R-R values wherein a cardiac cycle with an R-R value less than 80% of a mean R-R value is deemed to be a PVC. PVCs are counted and compared against a PVC threshold (that may depend on heart rate.) If the PVC count exceeds the threshold (or the aforementioned shifted set stability is poor), the ventricular IEGM is deemed to be unstable, the ST event is rejected as a false detection and the ST monitor resets to ninety second monitoring. If the ST event is not rejected, the device responds accordingly.

System and method embodiments are described herein.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 illustrates pertinent components of a single-chamber pacer/ICD equipped with ST stability discrimination for distinguishing a true ischemia event from a false detection due to AF or other confounding factors;

FIG. 2 provides an overview of an ST stability discrimination method for use by the system of FIG. 1;

FIG. 3 illustrates an exemplary embodiment of the general technique of FIG. 2, which examines sets of shifted ST segments to assess ventricular IEGM stability;

FIG. 4 illustrates another exemplary embodiment of the general technique of FIG. 2, which additionally counts PVCs;

FIG. 5 is a simplified, partly cutaway view of the heart of a patient, illustrating the exemplary single-chamber pacer/ICD of FIG. 1, along with a single RV lead implanted in the heart of the patient;

FIG. 6 is a simplified, partly cutaway view of the heart of a patient, illustrating an alternative embodiment of the system of FIG. 1, wherein the pacer/ICD is a multi-chamber device equipped with three leads implanted in the heart of the patient; and

FIG. 7 is a functional block diagram of the pacer/ICD of FIG. 6, illustrating basic device circuit elements that provide cardioversion, defibrillation and/or pacing stimulation in four chambers of the heart.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators are used to refer to like parts or elements throughout.

### Overview of ST Stability Discrimination Systems and Methods

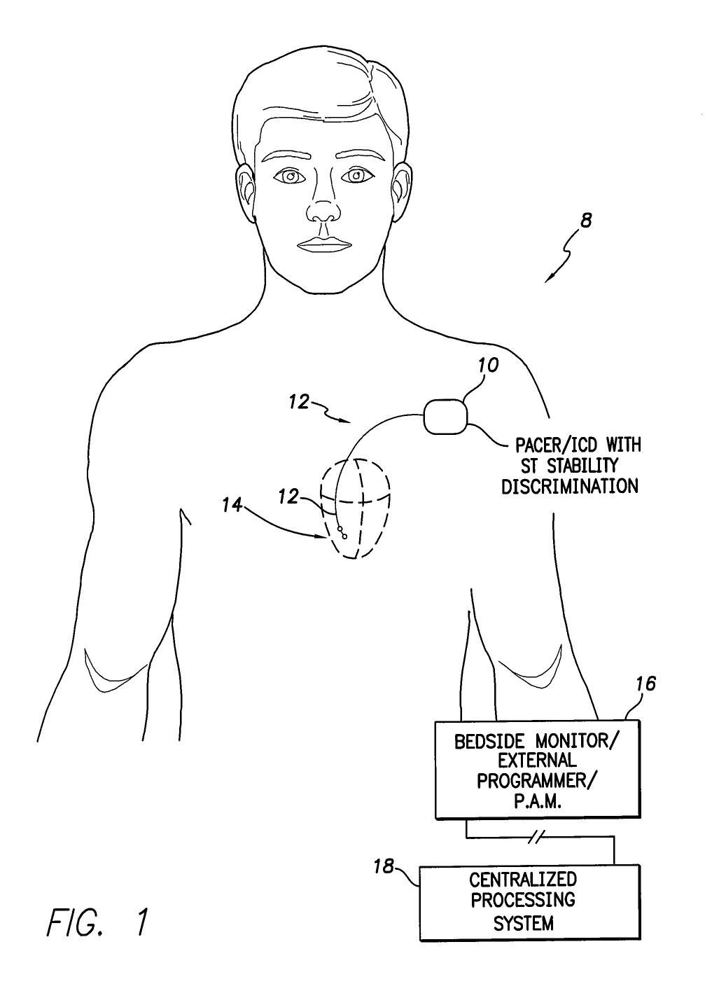

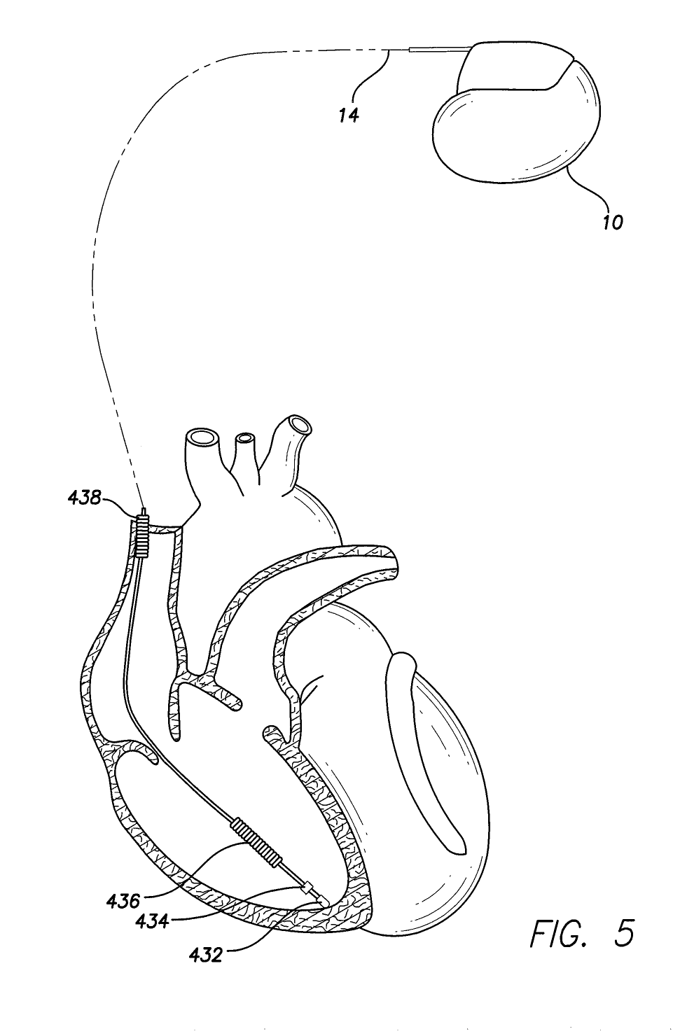

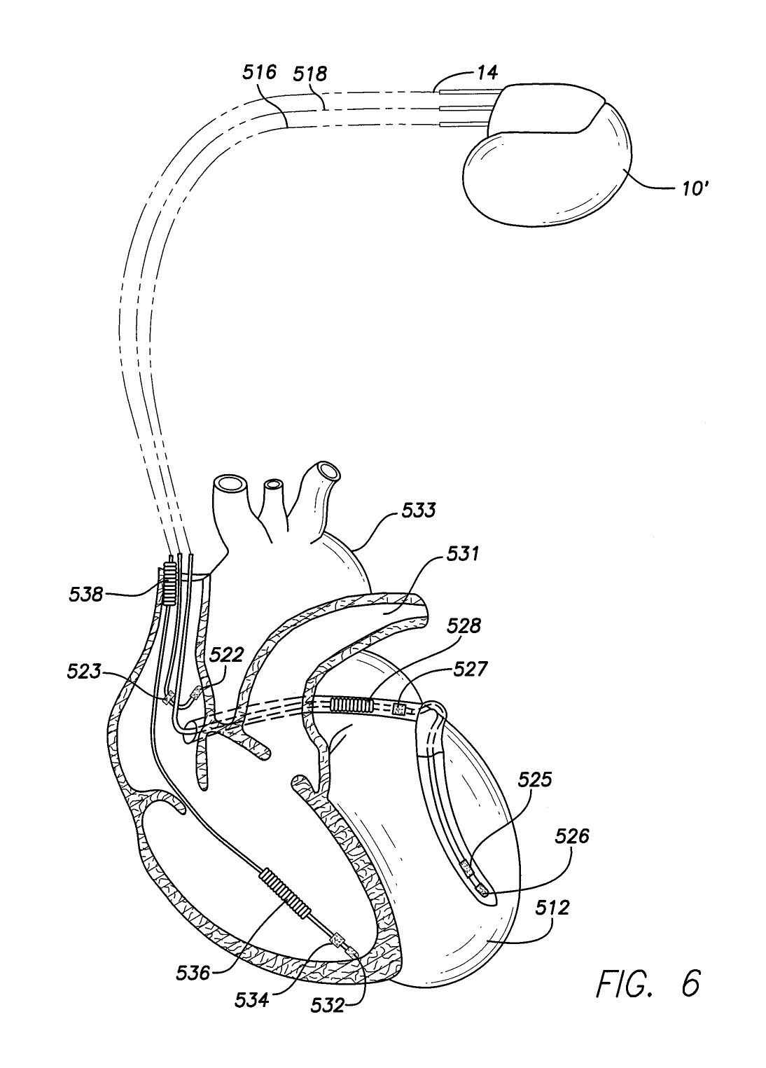

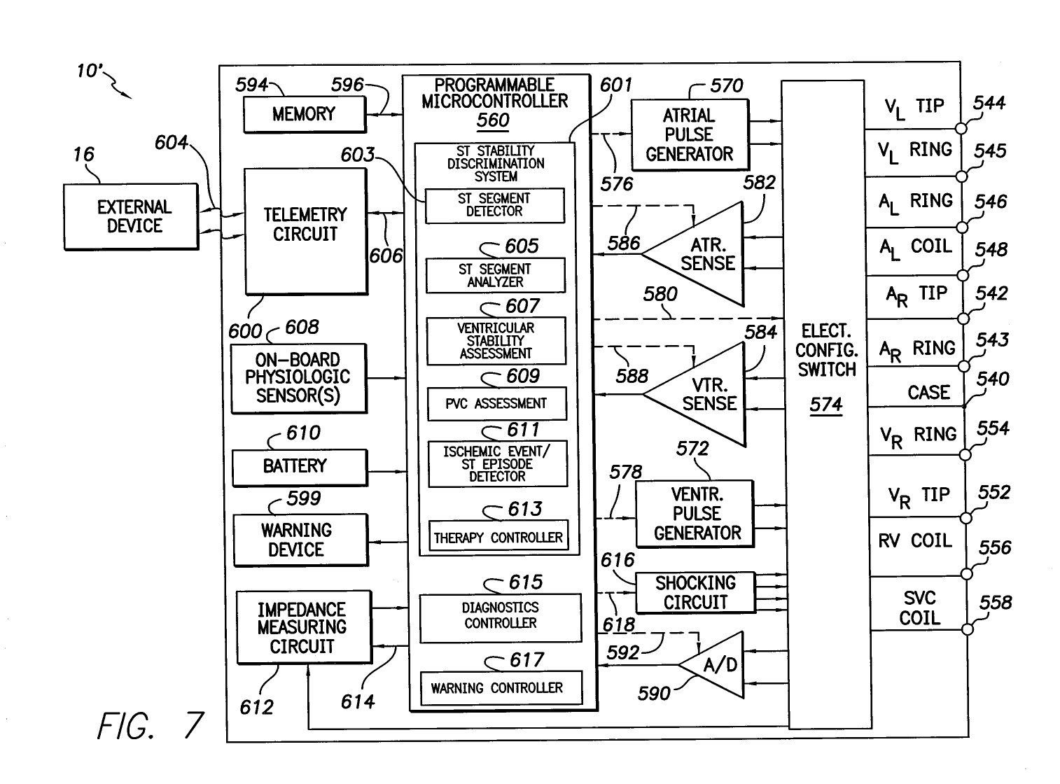

FIG. 1 illustrates an implantable medical system 8 having a pacemaker, ICD or other cardiac rhythm management device (CRMD) 10 equipped with an ST stability discrimination system for distinguishing true ischemic events such as ACS events from false events by analyzing signals sensed via an RV lead 12. In this example, the RV lead has only a bipolar tip/ring electrode pair for sensing a ventricular IEGM, i.e. the system is a single-chamber system not equipped for directly sensing an atrial IEGM or for performing a mode switch in response to AF. Note that the lead of FIG. 1 is shown in stylized form. A more complete and accurate illustration of an exemplary RV lead is illustrated in FIG. 5, described below, which additionally includes shock coil electrodes. Note also that, although the ST stability discrimination techniques described herein are primarily intended for use with single-chamber systems, at least some of the techniques are applicable to dual-or multi-chamber devices and so a more complete lead system including RA and LV leads is also described herein. (See FIGS. 6 and 7, discussed below.)

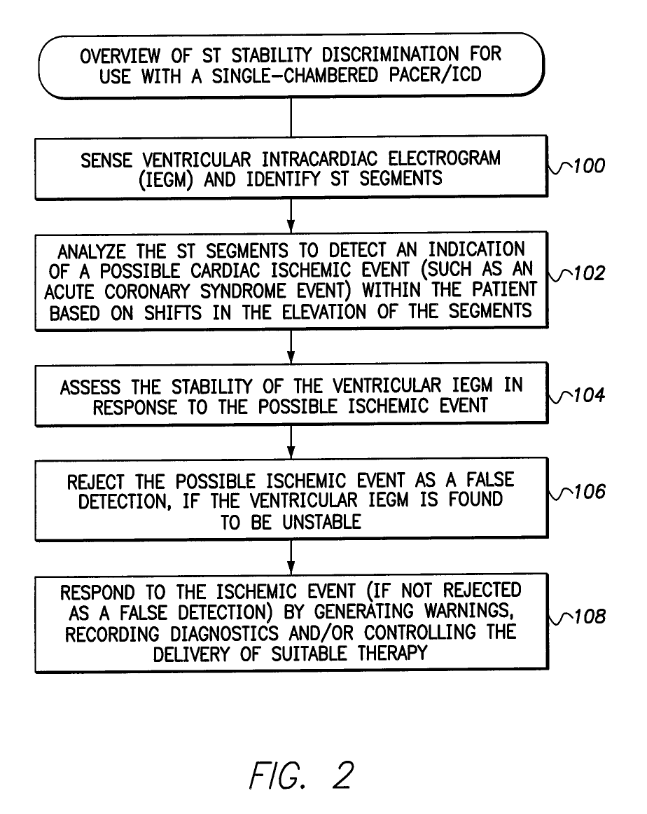

FIG. 2 summarizes an ST stability discrimination procedure that may be employed by the pacer/ICD of FIG. 1. Initially, at step 100, the implantable device senses ventricular IEGM and identifies ST segments therein, i.e. the interval between the end of the QRS complex and the beginning of T-wave. At step 102, the device analyzes the ST segments to detect an indication of a possible cardiac ischemic event (such as an ACS event) within the patient based on shifts in the elevation of the ST segments. At step 104, the device assesses the stability of the ventricular IEGM in response to the possible ischemic event using techniques described in detail below. As will be explained, in some examples the device analyzes shift sets of ST segments to assess stability. In other examples, the device additionally or alternatively counts PVCs. In any case, at step 106, the device rejects the possible ischemic event as a false detection if the ventricular IEGM is unstable (e.g. due to paroxysmal AF or frequent PVCs.) At step 108, if the ischemic event is not rejected as a false detection, the device responds by generating warnings, recording diagnostics and/or controlling the delivery of suitable therapy.

Referring again to FIG. 1, warning signals pertaining to ischemia or other conditions may be transmitted to a bedside monitor, external programmer, personal advisory module (PAM) 16 or other external system to alert the patient or caregiver, particularly if the event appears to be severe (such as an AMI.) The external system can also forward warning signals or other suitable information via a centralized processing system 18 to the patient's primary care physician or to emergency personnel. The centralized system may include such systems as the HouseCall™ remote monitoring system or the Merlin@home/Merlin.Net systems of St. Jude Medical. Warnings may also be generated using an internal warning device provided within the pacer/ICD. The internal warning device can be a vibrating device or a “tickle” voltage device that, in either case, provides perceptible stimulation to alert the patient. In addition, diagnostic information pertaining to the ischemic event may be stored within the implantable device for subsequent transmission to an external programmer for review by a clinician during a follow-up session between patient and clinician. The clinician may then prescribe appropriate therapies including medication regimes. The clinician may also adjust the operation of the implanted device to activate, deactivate or otherwise control any therapies automatically provided by the device.

Although not shown in FIG. 1, the implantable system may be equipped with an implantable drug pump or drug infusion device capable of the delivering medications directly to patient tissues. If so equipped, suitable medications might be delivered to the patient by the implantable system in response to ischemia, such as anti-thrombolytics. Implantable drug pumps for use in dispensing medications are discussed in U.S. Pat. No. 5,328,460 to Lord et al., entitled “Implantable Medication Infusion Pump Including Self-Contained Acoustic Fault Detection Apparatus.” (This patent also discusses implantable “tickle” warning devices that may be used to deliver warning signals.)

Thus, FIGS. 1 and 2 provide an overview of an exemplary implantable system and method. Embodiments may be provided that do not necessarily perform all of the described functions. For example, embodiments may be implemented that detect ischemia and generate warnings but which do not automatically deliver therapy in response to the ischemia. Drug pumps are not necessarily implanted. Bedside monitors or PAMs are not necessarily used. Some implementations may employ some form of external device for generating warning signals but no internal warning device. These are just a few exemplary embodiments. No attempt is made herein to describe all possible combinations of components that may be provided in accordance with the general principles of the invention. For brevity, implantable medical device 10 will be referred to herein as a “pacer/ICD” but it should be understood that it might additionally or alternatively provide CRT functions and hence may comprise a CRT-P or a CRT-D device or other CRMD. Note also that the particular shapes, sizes and locations of the implanted components shown in FIG. 1 are merely illustrative and may not necessarily correspond to actual implant locations. Implant locations for the leads are more precisely illustrated in FIGS. 5 and 6.

ST Stability Discrimination Based on ST Shifted Sets

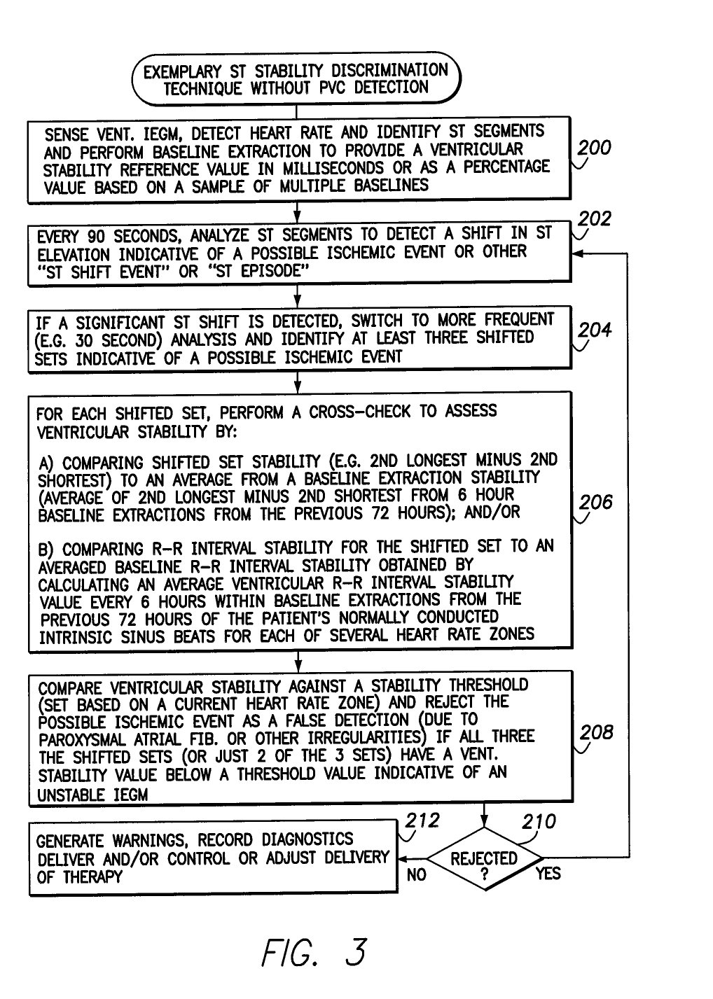

FIG. 3 illustrates an exemplary ST stability discrimination technique without PVC detection. At step 200, the pacer/ICD senses ventricular IEGM signals, detects the ventricular heart rate and identifies ST segments within the ventricular IEGM. The ventricular IEGM may be sensed, for example, using bipolar sensing via the tip and ring electrodes of the RV lead. In other examples, an integrated bipolar lead might be used with sensing between RV tip and RV coil. ST segments may be identified using any suitable technique. (Note that, in a typical exemplary device, ST monitoring operates independently form the standard ventricular sensing input used for bradycardiac therapy and tachycardiac discrimination. ST uses a dedicated high fidelity sense channel using the can to RV tip vector.) ST segments are discussed in U.S. Patent Application 2011/0245699 of Snell et al., entitled “Systems and Methods Related to ST Segment Monitoring by an Implantable Medical Device.” Also, at step 200, the pacer/ICD performs a baseline extraction to provide a ventricular stability reference value (in milliseconds or as a percentage value) based on a sample of multiple baselines. An exemplary baseline extraction technique is described in the patent application of Snell et al., which is incorporated by reference herein at least insofar as its descriptions of baseline extraction are concerned. Briefly, as described therein, an ST monitoring baseline extraction module periodically obtains baseline sets. A baseline set is a qualified reference of the patient's normal, non-shifted rhythm. The qualified reference can then be used to determine how a current ST shift deviates from the non-shifted rhythm. In one example, every six hours the ST monitoring baseline extraction module attempts to extract a reference ST segment during normal heart rhythm activity. Sets of up to fifteen beats are analyzed to classify each beat as a baseline beat or a non-baseline beat. A set is characterized as a baseline set if it contains an acceptable number of baseline beats, which meet various criteria described in the Snell et al. patent application.

At step 202, every ninety seconds (or other suitable periodic interval); the pacer/ICD analyzes the ST segments to detect a shift in ST elevation indicative of a possible ischemic event or other “ST episode.” ST shifts may be detected using any suitable technique. ST shifts are discussed in the Snell et al. patent application and in U.S. Pat. No. 7,725,171 to Zhu et al., entitled “System and Method for Tracking ST Shift Data Utilizing Histograms” and also in U.S. Pat. No. 7,949,388 of Fong, entitled “Methods and Systems to Characterize ST Segment Variation Over Time.” At step 204, if a significant ST shift is detected, the pacer/ICD switches to a more frequent (e.g. 30 second) analysis to identify at least three shifted sets of ST segments indicative of possible ischemia. A “set” may comprise all of the ST segments detected within the most recent thirty-second interval. (Note that, in at least some examples, a “shifted set” consists of at least eight and no more than fifteen “good” beats. Six of those eight beats must be shifted. It takes three “consecutive” shifted sets to declare an ST shift episode. If not, processing returns to step 202 for further ninety-second processing. Note also that a “good” beat is an intrinsic, non-PVC beat within a predetermined satisfactory heart rate zone. All other beats, including PVC beats, paced beats, and out-of-range beats are considered “bad” beats and are non-classified.

Assuming that at least three shifted sets are detected, the pacer/ICD at step 206 then analyzes each shifted set to assess ventricular stability. In the example of FIG. 3, two different techniques are illustrated, which may be performed alternatively or in combination, depending upon device programming. In the first technique, the pacer/ICD assesses ventricular stability by comparing shifted set stability (e.g. 2nd longest R-R interval minus the 2nd shortest R-R interval) to an average R-R interval obtained from the aforementioned baseline extraction (e.g. average of 2nd longest minus 2nd shortest from six hour baseline extractions obtained from the previous seventy-two hours.) If the “shifted sets” are unstable and greater than a programmed threshold (in ms or as a percentage value), the ST episode event is considered irregular/AF and no ST episode (e.g. no ischemic episode) is declared. In a second technique, the pacer/ICD compares R-R interval stability for the shifted set to an averaged baseline R-R interval stability obtained by calculating an average ventricular R-R interval stability value every six hours within the baseline extractions from the previous seventy-two hours of the patient's normally conducted intrinsic sinus beats for each of several heart rate zones, which may be in the range of 40 beats per minute (bpm) to 140 bpm. Once a shifted set occurs, the pacer/ICD compares the R-R interval stability for the shifted set to the averaged baseline R-R interval stability. For example, normally conducted ventricular beats would generate a stable calculation indicating a similarity to the baseline R-R interval stability, and thus a true ischemic episode. Ventricular stability is also discussed in U.S. Pat. No. 5,951,592 to Murphy, entitled “Apparatus and Method for Applying Antitachycardia Therapy based on Ventricular Stability” and in U.S. Pat. No. 5,480,413 to Greenhut et al., entitled “Apparatus and Method for Stabilizing the Ventricular Rate of a heart During Atrial Fibrillation.”

At step 208, the pacer/ICD compares the ventricular stability assessed using one of the aforementioned techniques against a suitable stability threshold (set based on the current heart rate zone) and rejects the possible ischemic event as a false detection (due to paroxysmal AF or other irregularities) if all three of the shifted sets have a ventricular stability value below a threshold value indicative of poor or unsatisfactory stability, i.e. the ventricular IEGM is deemed to be unstable. In other implementations, only two of the three sets with poor stability are sufficient to trigger a rejection of the event as a false detection. Whether the device requires that all shifted sets indicate poor stability or just two of three shifted sets is a programmable feature subject to clinician control. (Note also that, depending upon how ventricular stability is quantified, the stability threshold may instead represent a value above which the ventricular IEGM is deemed to be unstable. In that case, the device rejects the possible ischemic event as a false detection if the ventricular stability value exceeds the threshold indicating too much instability.)

If at step 210, the event is rejected, processing returns to step 202 where the procedure is reset to ninety-second processing. Assuming, though, that the event is not rejected, i.e. it is a true event, the device at step 212 generates warnings, records diagnostics and/or controls delivery of therapy. For example, pacing therapy may be adjusted in response to cardiac ischemia by, for example, reducing a base pacing rate to prevent a relatively high programmed base rate from exacerbating the ischemia. Anti-thrombolytics or other medications such as nitrates can be delivered using an implanted drug pump, if one is provided. Routine experimentation may be employed to identify medications for treatment of cardiac ischemia that are safe and effective for use in connection with an implantable drug pump. Although not specifically shown in FIG. 3, the procedure periodically returns to step 200 to perform a new baseline extraction.

Hence, using the technique of FIG. 3, a normally conducted (sinus) ventricular beat with regular conduction would generate a high ventricular stability score indicating a similarity to “Normal Sinus Rhythm” and would therefore be analyzable for ischemia. AF with irregular ventricular conduction would generate a low stability score and thus not be analyzable for ischemia. The ST stability discrimination procedure thereby operates as a cross-check once the pacer/ICD has detected an ST shift. If the ventricular stability value crosses the programmed threshold value, the rhythm is considered unstable and the device temporarily suspends ST monitoring thereby preventing the detection of a false positive ST episode and returns to once every ninety second monitoring of the ST segments. The technique is especially advantageous for use with single-chamber devices that do not provide mode switching in response to AF but, as noted above, the technique may also be exploited in dual- or multi-chamber devices as well, where appropriate.

ST Stability Discrimination Based on ST Shifted Sets and PVCs

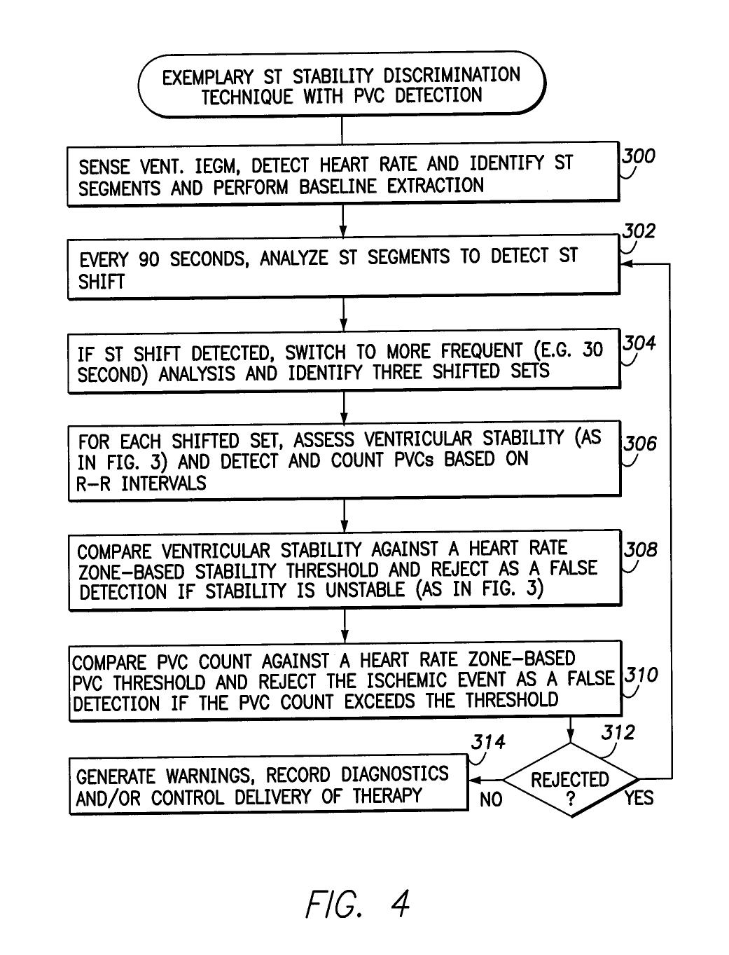

FIG. 4 illustrates an alternative discrimination technique that additionally exploits PVC detection. Many of the steps are similar to the technique of FIG. 3 and hence will not be described in detail again. Briefly, beginning at step 300, the pacer/ICD senses the ventricular IEGM, detects the ventricular rate, identifies ST segments and performs baseline extraction. At step 302, every ninety seconds (or other suitable periodic interval), the pacer/ICD analyzes the ST segments to detect a shift in ST elevation indicative of a possible ischemic event. At step 304, if a significant ST shift is detected, the pacer/ICD switches to the more frequent (e.g. 30 second) analysis to identify at least three shifted sets of ST segments indicative of possible ischemia. Assuming that at least three shifted sets are detected, the pacer/ICD at step 306 the analyzes each shifted set to assess ventricular stability as described above. Also at step 306, the device detects and counts PVCs based on R-R interval analysis or other suitable PVC detection techniques. The R-R interval analysis is preferably calculated on the beats within the shifted set to enhance PVC discrimination. That is, the R-R interval analysis is calculated on currently shifted sets. R-R intervals greater than or equal to 80% (of an R-R average) are declared a valid beat and any R-R interval less than 80% are defined as a PVC. PVC detection is also discussed in the aforementioned patent application to Snell et al. and in U.S. Pat. No. 5,097,832 to Buchanan, entitled “System and Method for Preventing False Pacemaker PVC Response.”

At step 308, the pacer/ICD compares ventricular stability against a suitable stability threshold (set based on the current heart rate zone) and rejects the possible ischemic event as a false detection the ventricular IEGM is unstable, as already discussed in connection with FIG. 3. At step 310, the device additionally compares the PVC count against a heart rate zone-based PVC threshold and rejects the ischemic event as a false detection if the PVC count exceeds the threshold. That is, when greater than an established number of PVCs are detected, the shifted set is discarded. The use of a PVCs count may be used in single-chamber embodiments and in dual- or multi-chamber embodiments as well. Note that although the examples provided herein illustrate PVC analysis in conjunction with shifted set stability analysis, it should be PVC analysis may be used as a standalone analysis technique. For example, a system might count PVCs and reject an ST episode if the PVC count exceeds its threshold, without necessarily also performing the shifted set stability analysis described in detail in FIG. 3.

Also, note that the ischemia detection techniques described herein may be used alone or in combination with other ischemia detection techniques, where appropriate. See, for example, ST techniques discussed in U.S. Pat. No. 6,108,577 to Benser, entitled “Method and Apparatus for Detecting Changes in Electrocardiogram Signals”; U.S. Pat. No. 7,610,086 to Ke et al., entitled “Ischemia Detection using T-wave Amplitude, QTmax and ST Segment Elevation and Pattern Classification Techniques”; and U.S. Pat. No. 7,225,015 to Min et al., entitled “System and Method for Detecting Cardiac Ischemia Based on T-Waves Using an Implantable Medical Device.” See, also, U.S. Pat. No. 7,756,572 to Fard et al., entitled “System and Method for Efficiently Distinguishing among Cardiac Ischemia, Hypoglycemia and Hyperglycemia using an Implantable Medical Device and an External System.”

Among the advantages of the techniques described herein, it is believed that there is generally reduced risk to patient and clinician secondary to False Positive Detection Rates causing unnecessary procedures secondary to AF and PVCs. Also, more control is provided over False Positive Detection Rates. This is particularly important given that AF is a common co-morbidity in coronary artery disease (CAD) patients. Moreover, the aforementioned techniques allow leveraging of ST monitoring technology to a large patient population with ischemia.

As noted, the techniques described herein can be implemented within a variety of implantable medical devices. For the sake of completeness, exemplary pacer/ICD implementations will now be described in detail.

### Exemplary Single-Chamber Pacer/ICD

With reference to FIGS. 5-7, exemplary pacer/ICDs will now be described. FIG. 5 provides a simplified block diagram of device 10, which in this example is a single-chamber device equipped with an RV lead 14 having a ventricular tip electrode 432, an RV ring electrode 434, an RV coil electrode 436 and a superior vena cava (SVC) coil electrode 438. Typically, the RV lead is transvenously inserted into the heart so as to place the RV coil 536 in the right ventricular apex and the SVC coil electrode 438 in the superior vena cava. Accordingly, the RV lead is capable of receiving cardiac signals and delivering stimulation in the form of pacing and shock therapy to the right ventricle. Device 10 is thereby capable of treating at least some arrhythmias with stimulation therapy, including cardioversion, defibrillation and pacing stimulation.

As noted, in at least some examples, aspects of the invention may be exploited by dual- or multi-chamber devices as well. Accordingly, FIG. 6 provides a diagram of a multi-chamber device 10′ equipped with an RV lead (having RV tip/ring electrodes 534 and 536 and coils 536 and 538) and additionally equipped with an RA lead and an LV lead. To provide right atrial chamber pacing stimulation and sensing, device 10′ is shown in electrical communication with a heart 512 by way of a right atrial lead 518 having an atrial tip electrode 522 and an atrial ring electrode 523 implanted in the atrial appendage. To sense left atrial and ventricular cardiac signals and to provide left chamber pacing therapy, device 10 is coupled to an LV lead 516 designed for placement in the “CS region” via the CS os for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary LV lead 516 is designed to receive atrial and ventricular cardiac signals and to deliver left ventricular pacing therapy using a pair of left ventricular electrodes 526 and 525, left atrial pacing therapy using at least a left atrial ring electrode 527 and shocking therapy using at least a left atrial coil electrode 528 implanted on or near the left atrium. In other examples, more or fewer LV electrodes are provided. Although only three leads are shown in FIG. 6, it should be understood that additional leads (with one or more pacing, sensing and/or shocking electrodes) might be used and/or additional electrodes might be provided on the leads already shown.

A simplified block diagram of internal components of device 10′ is shown in FIG. 7. While a particular device is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. The housing 540 for device 10′, shown schematically in FIG. 11, is often referred to as the “can,” “case” or “case electrode” and may be programmably selected to act as the return electrode for all “unipolar” modes. The housing 540 may further be used as a return electrode alone or in combination with one or more of the coil electrodes, 528, 536 and 538, for shocking purposes. The housing 540 further includes a connector (not shown) having a plurality of terminals, 542, 543, 544, 545, 546, 548, 552, 554, 556 and 558 (shown schematically and, for convenience, the names of the electrodes to which they are connected are shown next to the terminals). As such, to achieve right atrial sensing and pacing, the connector includes at least a right atrial tip terminal (A_{R} TIP) 542 adapted for connection to the atrial tip electrode 522 and a right atrial ring (A_{R} RING) electrode 543 adapted for connection to right atrial ring electrode 523. To achieve left chamber sensing, pacing and shocking, the connector includes a left ventricular tip terminal (V_{L} TIP) 544 and a left ventricular ring terminal (V_{L} RING) 545.

The connector also includes a left atrial ring terminal (A_{L} RING) 546 and a left atrial shocking terminal (A_{L} COIL) 548, which are adapted for connection to the left atrial ring electrode 527 and the left atrial coil electrode 528, respectively. To support right chamber sensing, pacing and shocking, the connector further includes a right ventricular tip terminal (V_{R} TIP) 552, a right ventricular ring terminal (V_{R} RING) 554, a right ventricular shocking terminal (V_{R} COIL) 556, and an SVC shocking terminal (SVC COIL) 558, which are adapted for connection to the right ventricular tip electrode 532, right ventricular ring electrode 534, the V_{R} coil electrode 536, and the SVC coil electrode 538, respectively.

At the core of pacer/ICD 10′ is a programmable microcontroller 560, which controls the various modes of stimulation therapy. As is well known in the art, the microcontroller 560 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 560 includes the ability to process or monitor input signals (data) as controlled by a program code stored in a designated block of memory. The details of the design and operation of the microcontroller 560 are not critical to the invention. Rather, any suitable microcontroller 560 may be used that carries out the functions described herein. The use of microprocessor based control circuits for performing timing and data analysis functions are well known in the art.

As shown in FIG. 7, an atrial pulse generator 570 and a ventricular pulse generator 572 generate pacing stimulation pulses for delivery by the right atrial lead 518, the right ventricular lead 14 and/or the LV/CS lead 516 via an electrode configuration switch 574. It is understood that in order to provide stimulation therapy in each of the four chambers of the heart, the atrial and ventricular pulse generators, 570 and 572, may include dedicated, independent pulse generators, multiplexed pulse generators or shared pulse generators. The pulse generators, 570 and 572, are controlled by the microcontroller 560 via appropriate control signals, 576 and 578, respectively, to trigger or inhibit the stimulation pulses.

The microcontroller 560 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 574 includes a plurality of switches for connecting the desired electrodes to the appropriate I/O circuits, thereby providing complete electrode programmability. Accordingly, the switch 574, in response to a control signal 580 from the microcontroller 560, determines the polarity of the stimulation pulses (e.g., unipolar, bipolar, combipolar, etc.) by selectively closing the appropriate combination of switches (not shown) as is known in the art.

Atrial sensing circuits 582 and ventricular sensing circuits 584 may also be selectively coupled to the right atrial lead 518, LV/CS lead 516, and the right ventricular lead 12, through the switch 574 for detecting the presence of cardiac activity in each of the four chambers of the heart. Accordingly, the atrial (ATR. SENSE) and ventricular (VTR. SENSE) sensing circuits, 582 and 584, may include dedicated sense amplifiers, multiplexed amplifiers or shared amplifiers. The switch 574 determines the “sensing polarity” of the cardiac signal by selectively closing the appropriate switches, as is also known in the art. In this way, the clinician may program the sensing polarity independent of the stimulation polarity. Each sensing circuit, 582 and 584, preferably employs one or more low power, precision amplifiers with programmable gain and/or automatic gain control, bandpass filtering, and a threshold detection circuit, as known in the art, to selectively sense the cardiac signal of interest. The automatic gain control enables pacer/ICD 10′ to deal effectively with the difficult problem of sensing the low amplitude signal characteristics of atrial or ventricular fibrillation. The outputs of the atrial and ventricular sensing circuits, 582 and 584, are connected to the microcontroller 560 which, in turn, are able to trigger or inhibit the atrial and ventricular pulse generators, 570 and 572, respectively, in a demand fashion in response to the absence or presence of cardiac activity in the appropriate chambers of the heart.

For arrhythmia detection, pacer/ICD 10′ utilizes the atrial and ventricular sensing circuits, 582 and 584, to sense cardiac signals to determine whether a rhythm is physiologic or pathologic. As used in this section, “sensing” is reserved for the noting of an electrical signal, and “detection” is the processing of these sensed signals and noting the presence of an arrhythmia. The timing intervals between sensed events (e.g., AS, VS, and depolarization signals associated with fibrillation which are sometimes referred to as “F-waves” or “Fib-waves”) are then classified by the microcontroller 560 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (ND) data acquisition system 590. The data acquisition system 590 is configured to acquire intracardiac electrogram signals, convert the raw analog data into a digital signal, and store the digital signals for later processing and/or telemetric transmission to an external device 16. The data acquisition system 590 is coupled to the right atrial lead 518, the LV/CS lead 516, and the right ventricular lead 14 through the switch 574 to sample cardiac signals across any pair of desired electrodes. The microcontroller 560 is further coupled to a memory 594 by a suitable data/address bus 596, wherein the programmable operating parameters used by the microcontroller 560 are stored and modified, as required, in order to customize the operation of pacer/ICD 10′ to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10′ may be non-invasively programmed into the memory 594 through a telemetry circuit 600 in telemetric communication with the external device 16, such as a programmer, transtelephonic transceiver or a diagnostic system analyzer. The telemetry circuit 600 is activated by the microcontroller by a control signal 606. The telemetry circuit 600 advantageously allows intracardiac electrograms and status information relating to the operation of pacer/ICD 10′ (as contained in the microcontroller 560 or memory 594) to be sent to the external device 16 through an established communication link 604. The external device 16 may alternatively be a bedside monitor or PAM, as already discussed.

Pacer/ICD 10′ further includes an on-board accelerometer or other physiologic sensor 608, sometimes referred to as a “rate-responsive” sensor because it is typically used to adjust pacing stimulation rate according to the exercise state of the patient. However, physiological or hemodynamic sensor(s) 608 can be equipped to sense any of a variety of cardiomechanical parameters, such as heart sounds, systemic pressure, etc. As can be appreciated, at least some these sensors may be mounted outside of the housing of the device and, in many cases, will be mounted to the leads of the device. Moreover, the physiological sensor 608 may further be used to detect changes in cardiac output, changes in the physiological condition of the heart, or diurnal changes in activity (e.g., detecting sleep and wake states) and to detect arousal from sleep. Accordingly, the microcontroller 560 responds by adjusting the various pacing parameters (such as rate, AV delay, V-V delay, etc.) at which the atrial and ventricular pulse generators, 570 and 572, generate stimulation pulses. While shown as being included within pacer/ICD 10′, it is to be understood that physiologic/hemodynamic sensors may also be external to pacer/ICD 10′, yet still be implanted within or carried by the patient. This is shown by way of physiological/hemodynamic sensor(s) 611. A common type of internal rate responsive sensor is an activity sensor incorporating an accelerometer or a piezoelectric crystal and/or a 3D-accelerometer capable of determining the posture within a given patient, which is mounted within the housing 540 of pacer/ICD 10′. Other types of physiologic sensors are also known, for example, sensors that sense the oxygen content of blood, respiration rate and/or minute ventilation, pH of blood, ventricular gradient, etc.,

The pacer/ICD additionally includes a battery 610, which provides operating power to all of the circuits shown in FIG. 7. The battery 610 may vary depending on the capabilities of pacer/ICD 10′. If the system only provides low voltage therapy, a lithium iodine or lithium copper fluoride cell typically may be utilized. For pacer/ICD 10′, which employs shocking therapy, the battery 610 should be capable of operating at low current drains for long periods, and then be capable of providing high-current pulses (for capacitor charging) when the patient requires a shock pulse. The battery 610 should also have a predictable discharge characteristic so that elective replacement time can be detected. Accordingly, appropriate batteries are employed.

As further shown in FIG. 7, pacer/ICD 10′ is shown as having an impedance measuring circuit 612, which is enabled by the microcontroller 560 via a control signal 614. Uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; measuring intracardiac impedance; measuring respiration; and detecting the opening of heart valves, etc. The impedance measuring circuit 612 is coupled to the switch 674 so that any desired electrode may be used.

In the case where pacer/ICD 10′ is intended to operate as an implantable cardioverter/defibrillator (ICD) device, it detects the occurrence of an arrhythmia requiring a shock and automatically applies appropriate electrical shock therapy to the heart aimed at terminating the detected arrhythmia. To this end, the microcontroller 560 further controls a shocking circuit 616 by way of a control signal 618. The shocking circuit 616 generates shocking pulses of low (up to 0.5 joules), moderate (0.5-10 joules) or high energy (11 to 40 joules or more), as controlled by the microcontroller 560. Such shocking pulses are applied to the heart of the patient through at least two shocking electrodes, and as shown in this embodiment, selected from the left atrial coil electrode 528, the RV coil electrode 536, and/or the SVC coil electrode 538. The housing 540 may act as an active electrode in combination with the RV electrode 536, or as part of a split electrical vector using the SVC coil electrode 538 or the left atrial coil electrode 528 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 6-40 joules), delivered asynchronously (since R-waves may be too disorganized), and pertaining exclusively to the treatment of fibrillation. Accordingly, the microcontroller 560 is capable of controlling the synchronous or asynchronous delivery of the shocking pulses.

Insofar as ST stability discrimination is concerned, the microcontroller includes an ST stability discrimination system 601 operative to control the stability discrimination based on ventricular IEGM signals received from ventricular sense amplifier 584. In the example of FIG. 7, the ST stability discrimination system includes an ST segment detector 603 operative to identify ST segments within the ventricular IEGM and an ST segment analyzer 605 operative to analyze the ST segments to detect an indication of a possible ischemic event within the patient. A ventricular stability assessment system 607 is operative in response to the possible ischemic event to assess ventricular stability within the patient using techniques described above. A PVC assessment system 609 detects and counts PVCs. An ischemic event detection controller 611 (i.e. an ST episode controller) is operative to reject the possible ischemic event as a false detection if the ventricular IEGM is deemed too unstable. A therapy controller 613 controls or adjusts delivery of therapy in response to the event. A diagnostics controller 615 controls storage of diagnostics data in memory 594. A warning controller 617 controls the generation of warning signals using warning device 599 or by controlling the telemetry circuit to transmit suitable warning signals to external device 16.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

In general, while the invention has been described with reference to particular embodiments, modifications can be made thereto without departing from the scope of the invention. Note also that the term “including” as used herein is intended to be inclusive, i.e. “including but not limited to.”

1. A method for use with an implantable medical device for implant within a patient, the method comprising:
inputting a ventricular stability reference value derived from a baseline extraction;
sensing a ventricular intracardiac electrogram (IEGM) signal and identifying ST segments within the ventricular IEGM signal;
analyzing the ST segments to detect an indication of a possible cardiac ischemic event within the patient;
in response to the detection of a possible ischemic event detecting shifted sets of ST intervals and detecting intervals between consecutive depolarization events (R-R intervals) within the shifted sets; and
comparing R-R interval stability for the shifted sets to an averaged baseline R-R interval stability obtained from the baseline extractions to detect one or more unstable shifted sets;
rejecting the possible ischemic event as a false detection as a function of the comparison of the R-R interval stability for the shifted sets and the baseline extractions; and
responding to the ischemic event if not rejected as a false detection.

2. The method of claim 1 wherein the ST segments represent a portion of the ventricular IEGM between an end of depolarization event (QRS complex) and a beginning of a repolarization event (T-wave).

3. The method of claim 1 wherein analyzing ST segments to detect an indication of a possible ischemic event includes detecting a shift in ST segment elevation over time.

4. The method of claim 3 wherein analyzing ST segments to detect an indication of a possible ischemic event is performed relatively less frequently while no significant ST shift is detected and then is performed relatively more frequently if a significant ST shift is detected.

5. The method of claim 4 wherein analyzing ST segments to detect an indication of a possible ischemic event is performed about every ninety seconds while no significant ST shift is detected and then is performed about every thirty seconds if a significant ST shift is detected.

6. The method of claim 4 wherein, if the possible ischemic event is rejected as a false detection, then analysis of additional ST segments to detect an indication of another possible ischemic event is performed relatively less frequently.

7. The method of claim 1 wherein the ischemic event is an acute coronary syndrome (ACS) event.

8. The method of claim 1 wherein assessing the stability of the ventricular IEGM in response to the possible ischemic event includes determining a value representative of ventricular stability.

9-10. (canceled)

11. The method of claim 1 wherein rejecting the possible ischemic event as a false detection if the ventricular IEGM signal is unstable includes:
comparing the value representative of ventricular stability to an acceptable stability threshold; and
rejecting the possible ischemic event as a false detection if the value representative of ventricular stability crosses the stability threshold.

12. The method of claim 11 wherein a plurality of values representative of ventricular stability are determined and the ischemic event is rejected only if all of the values cross the threshold.

13. The method of claim 11 wherein a plurality of values representative of ventricular stability are determined and the ischemic event is rejected if a predetermined number of the values cross stability threshold.

14. The method of claim 13 wherein three sets of values representative of ventricular stability are determined and the ischemic event is rejected if at least two of the three sets of values cross the stability threshold.

15. The method of claim 1 further including detecting premature ventricular contractions (PVCs) and rejecting the possible ischemic event as a false detection in response to an excess of PVCs.

16. The method of claim 15 wherein the possible ischemic event is rejected as a false detection if a count of PVCs exceeds a permissible threshold.

17. The method of claim 1 further including determining a heart rate zone of the patient and wherein assessing stability of the ventricular IEGM signal distinguishes among heart rates zones.

18. The method of claim 17 wherein assessing the stability of the ventricular IEGM signal exploits stability thresholds and wherein different stability thresholds are used within different heart rate zones.

19. The method of claim 1 wherein the implantable medical device is a single-chamber device and wherein the method is performed based on ventricular IEGM signals sensed using a right ventricular (RV) lead of the signal-chamber device.

20. The method of claim 1 wherein the implantable medical device is a dual-chamber device and wherein the method is performed based on ventricular IEGM signals sensed using an RV lead of the dual-chamber device.

21. A system for use with an implantable medical device for implant within a patient, the system comprising:
a ventricular intracardiac electrogram (IEGM) signal sensing system operative to sense a ventricular IEGM signal;
an ST segment detector operative to identify ST segments within the ventricular IEGM;
an ST segment analyzer operative to analyze ST segments to detect an indication of a possible ischemic event within the patient;
a ventricular stability assessment system operative in response to the possible ischemic event to detect shifted sets of ST intervals and analyze intervals between consecutive depolarization events (R-R intervals) within the shifted sets, the ventricular stability assessment system being further operative to compare R-R interval stability for the shifted sets to an averaged baseline R-R interval stability obtained from baseline extractions to detect one or more unstable shifted sets; and
an ischemic event detection controller operative to reject the possible ischemic event as a false detection if the ventricular IEGM signal is unstable and further operative to respond to the ischemic event if not rejected as a false detection.

22. A system for use with an implantable medical device for implant within a patient, the system comprising:
means for sensing a ventricular intracardiac electrogram (IEGM) signal and identifying ST segments within the ventricular IEGM signal;
means for analyzing the ST segments to detect an indication of a possible cardiac ischemic event within the patient;
means for detecting shifted sets of ST intervals and detecting intervals between consecutive depolarization events (R-R intervals) within the shifted sets in response to the possible ischemic event;
means for comparing R-R interval stability for the shifted sets to an averaged baseline R-R interval stability obtained from the baseline extractions to detect one or more unstable shifted sets;
means for rejecting the possible ischemic event as a false detection if the ventricular IEGM signal is unstable; and
means for responding to the ischemic event if not rejected as a false detection.
